(19) United States
(12) Patent Application Publication
(10) Pub. No.: US 2026/0200888 A1
(43) Pub. Date: Jul. 16, 2026

(54) BIO-BASED COMPOUNDS

(71) Applicant: SPECIALTY OPERATIONS FRANCE, Lyon (FR)

(72) Inventors: Olivier Back, Saint-Fons (FR); Rawad Tadmouri, Pessac (FR)

(73) Assignee: SPECIALTY OPERATIONS FRANCE, Lyon (FR)

(21) Appl. No.: 19/134,900

(22) PCT Filed: Dec. 4, 2023
(86) PCT No.: PCT/EP2023/084185

§ 371 Date: Jun. 2, 2025

(30) Foreign Application Priority Data
Dec. 2, 2022 (EP) 22211266.6

(51) Int. Cl. C07D 317/30; A01N 25/02; A01N 35/10; A01N 43/54; A01N 43/56; A01N 43/653; A01P 3/00

(52) U.S. Cl. CPC C07D 317/30; A01N 25/02; A01N 35/10; A01N 43/54; A01N 43/56; A01N 43/653; A01P 3/00

(57) ABSTRACT

A compound of formula (I) includes R₁ and R₂ that are independently selected from —(C₁-C₆)alkyl including (C₁)alkyl, —(C₂)alkyl, —(C₃)alkyl, —(C₄)alkyl, —(C₅)alkyl, and —(C₆)alkyl, which can be optionally interrupted and/or substituted by a heteroatom or heteroatom-containing group, or R1 and R2 form with the nitrogen atom a heterocycle containing from 4 to 8 atoms. The compound of formula (I) also includes R₃, which is hydrogen, or —(C₁-C₄)alkyl, and can optionally be interrupted and/or substituted by a heteroatom or heteroatom containing group. The compound of formula (I) has the proviso that when R₁ and R₂ are —CH₃, then R₃ is not hydrogen.

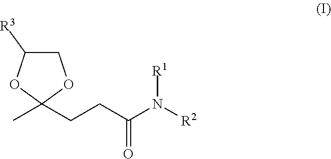

### TECHNICAL FIELD

The present invention relates to bio-based compounds, suitable as biodegradable and eco-friendly solvents, in particular in agrochemicals.

### TECHNICAL BACKGROUND

Industry uses many chemical compounds as solvents, for example for preparing chemicals and materials, for formulating chemical compounds, or for treating surfaces. For example, solvents are used for the formulation of agricultural compounds, in particular phytosanitary active agents (fertilizers, pesticides . . . ), for instance in the form of emulsifiable concentrates (ECs) intended to be diluted in water by the farmer before being applied to a field.

In the case of fertilizers as active agents, nitrogen fertilizer stabilizers, such as urease or nitrification inhibitors are generally applied together with the fertilizer as enhanced efficiency fertilizer (EEF) into or onto the soil. This ensures that the stabilizer comes into contact, together with the fertilizer, with the soil. The nitrogen fertilizer stabilizers are dissolved in a polar solvent and can be incorporated in the fertilizer by adding this solution into the melt prior to granulation or prilling of the fertilizer. A process of this kind is described for urease inhibitors in U.S. Pat. No. 5,352,265, for example. A further option is to apply the fertilizer stabilizer, such as the urease inhibitor, as a coating to the granules or prills. In these processes, solvents such as DMSO (dimethylsulfoxide), NMP (N-methyl 2-pyrrolidone), DMF (N,N-dimethyl formamide), DMAc (N,N-dimethyl acetamide) or glycol-based solvents are used. These solvents are, however, environmentally harmful, have poor biodegradability profiles while being very mobile (because highly water soluble) in the environment and are potentially toxic for humans or small organisms.

The industry in the field of agriculture attempts to achieve a concentration of the agricultural active compound(s) as high as possible in the respective formulation since a high concentration of the agricultural compound(s) allows the volumes to be applied to be reduced and, as a consequence, entails savings with regard to the adjuvant materials applied and with regard to packaging and logistics. Highly-concentrated stable formulations and coformulations with environmentally friendly adjuvants are therefore of interest as a matter of principle.

For agricultural active compounds with a low or relatively low water solubility, the use of appropriate solvents is especially interesting to prepare concentrated liquid formulations, in the form of emulsifiable concentrates (EC), concentrated emulsions in water (EW), microemulsions (ME), suspoemulsions (SE), oil dispersions (OD), dispersible concentrates (DC).

Such concentrated formulations of agricultural compounds are generally diluted prior to agricultural use. The dilution effected by the farmer is generally performed by mixing the agrochemical formulation with water.

In addition, certain solid agricultural active compounds are often difficult to formulate. For certain agricultural compounds, it is difficult to produce concentrated formulations that are easy for the farmer to dilute, stable and free of substantial drawbacks (real or perceived) with regard to safety, toxicity and/or ecotoxicity. For certain agricultural compounds, it is difficult to formulate at relatively high concentrations with sufficient stability. In particular, it is necessary to avoid the appearance of crystals, in particular at low temperature and/or during dilution and/or during storage of the composition, in particular at low temperature. The crystals may have harmful effects, especially blocking the filters of the devices used for spreading the dilute composition, blocking the spraying devices, reducing the overall activity of the formulation, creating unnecessary problems of waste-management procedures for removing the crystals, and/or causing poor distribution of the agricultural material(s) on the agricultural field.

More generally, industry is looking for novel compounds that make it possible to vary or optimize the products and processes in which solvents, especially polar solvents, are to be used. In particular, the agrochemical industry is constantly looking for new solvents and solvent compositions having properties that are satisfactory for agricultural application, like for example, good solubilization efficiency. In addition, the cost of the solvent compositions should generally be modest, and preferably they should have a favourable toxicology and/or ecotoxicology profile, in particular low toxicity and/or low hazard potential, and/or low volatility (low VOC—volatile organic compounds) and/or advantageously high degree of biodegradability and/or renewability.

Therefore, it is an object of the present invention to provide a preferably bio-based solvent that is advantageously non-toxic, environmentally safe to use, preferably with a good biodegradability profile and is in particular suitable for all applications mentioned above, in particular to dissolve polar agricultural compounds, such as pesticides and fertilizer stabilizers.

There are a limited number of eco-friendly polar solvents on the market, such as Cyrene® (Dihydrolevoglucosenone), Rhodiasolv® PolarClean, NBP (N-butyl-2-pyrrolidone)), N,N-dimethyl lactamide and Rhodiasolv® ADMA 10. However, these solvents are not always suitable for all purposes, including the dissolution of agricultural, polar active compounds such as fertilizer stabilizers at high concentration while generally also ensuring storage stability over a range of different temperatures.

Moreover, if the use of specific solvent systems based on a single solvent such as N-methyl-2-pyrrolidone (NMP) can enable the dissolution of a certain number of agricultural materials, it is however known as presenting reprotoxicity hazards, especially for the operatives and users handling it.

Also in other fields of applications, like polymer transformation or recycling, home and personal care, there is a need for multipurpose solvents which are eco-friendly.

Therefore, it is an object of the present invention to provide a preferably sustainable, bio-based and eco-friendly solvent that can advantageously dissolve polar compounds at high concentrations (e. g. pesticides and fertilizer stabilizers), while generally also ensuring storage stability over a range of different temperatures.

### SUMMARY OF THE INVENTION

These objects have surprisingly been solved by a compound of formula (I):

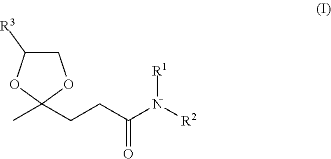

wherein
R¹ and R² are independently selected from —(C₁-C₆)alkyl including —(C₁)alkyl, —(C₂)alkyl, —(C₃)alkyl, —(C₄)alkyl, —(C₅)alkyl, and —(C₆)alkyl, optionally interrupted and/or substituted by a heteroatom or heteroatom-containing group, or R¹ and R² form with the nitrogen atom a heterocycle containing from 4 to 8 atoms; and
R³ is hydrogen, or —(C₁-C₄)alkyl, optionally interrupted and/or substituted by a heteroatom or heteroatom containing group;
with the proviso that when R¹ and R² are —CH₃, then R³ is not hydrogen.

Compounds of formula (I) where R¹ and R² are —CH₃ and R³ is hydrogen have been described as starting material for the production of molecules of interest in the field of medicinal chemistry (e.g. tetracyclines): “Synthesis of phenols by polyketide condensation and an efficient synthesis of 3,5-dioxoalkanoates from amides”, Yamaguchi et al., Chemistry Letters (1985), (8), 1145-8. In this paper, no details are given on the properties of this compound (physical properties, analytical data . . . ) nor on its synthesis.

The ketal-amide derivatives of levulinic acid according to the invention, i.e. the compounds of formula (I) defined above, are bio-based compounds that can be produced following a sustainable chemical process (i.e. advantageously displaying a very good atom economy and generating little waste) from levulinic acid, diols and secondary amine building blocks, which preferably can all be obtained by biological processes or from bio-based raw materials.

It has been found that the compounds of formula (I) are advantageously eco-friendly solvents, have preferably good safety tand sustainable profiles, with none or very low hazard classification and none or very low ecotoxicity, and are furthermore excellent solvents. It has been found that the compounds of the present invention are namely excellent solvents for dissolving pesticides, for example fungicides, herbicides and insecticides, and/or (nitrogen) fertilizer stabilizers (such as urease and nitrification inhibitors), and many other polar compounds. It has also been found that the compounds of the invention can also be used as solvent in many other applications like coating applications, for the manufacture of membranes, for the manufacture of solid batteries, for the recycling of polymers, for the cleaning of equipments, and in home and personal care compositions. These compounds are preferably biodegradable.

The present invention further relates to a method for production of the compounds of the present invention from levulinic acid or an ester or salt thereof, comprising at least the following steps in any order:
(a) ketalization with a compound of formula R³—CH(OH)CH₂OH
(b) amidification with an amine of formula HNR¹R²,
wherein R¹, R², and R³ are defined as above.

In an aspect, the invention relates to a composition comprising the compound of the present invention.

In another aspect, the present invention relates to the use of the compound of the present invention as a solvent, in particular in agriculture formulations for plant protection products, such as e.g. pesticides, for biostimulants, biologicals, plant growth regulator, and in enhanced efficiency fertilizer formulations containing an urease or nitrification inhibitor.

### DETAILED DESCRIPTION OF THE INVENTION

The following definitions are relevant in connection with the embodiments of the present invention.

The meaning of the term “comprising” is to be interpreted as encompassing all the specifically mentioned features as well optional, additional, unspecified ones, whereas the term “consisting of” only includes those features as specified. Therefore, “comprising” includes as a limiting case the composition specified by “consisting of”.

The term “wt. %” refers to the amount of the respective ingredient by weight based on the total amount of the composition, unless noted otherwise.

As used herein, the singular forms “a”, “an”, and “the” include both singular and plural referents unless the context clearly dictates otherwise. By way of example, “a pesticide” means one pesticide or more than one pesticide.

The term “(Cₓ-C_{y})alkyl” as used herein denotes in each case a straight-chain or branched alkyl group having from x to y carbon atoms. Examples of a (C₁-C₆)alkyl group are methyl, ethyl, n-propyl, iso-propyl, n-butyl, 2-butyl, iso-butyl, tert-butyl, n-pentyl, 1-methylbutyl, 2-methylbutyl, 3-methylbutyl, 2,2-dimethylpropyl, 1-ethylpropyl, 1,1-dimethylpropyl, 1,2-dimethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, and 1-ethyl-2-methylpropyl.

The term “alkoxy” as used herein denotes in each case a straight-chain or branched alkyl group which is bound via an oxygen, thus an alkoxy group may be represented as —O-alkyl. Examples of an alkoxy group are methoxy, ethoxy, n-propoxy, iso-propoxy, n-butyloxy, 2-butyloxy, iso-butyloxy, and tert-butyloxy.

The term “alkoxyalkyl” as used herein may be represented as -alkyl-O-alkyl and refers to an alkyl usually comprising 1 to 4, preferably 1 to 2 carbon atoms, wherein 1 carbon atom carries an alkoxy radical usually comprising 1 to 4, preferably 1 or 2 carbon atoms as defined above. Examples are —CH₂OCH₃, —CH₂—O—C₂H₅, 2-(methoxy)ethyl, and 2-(ethoxy)ethyl.

The term “alkyl substituted by a heteroatom” refers to an alkyl group, wherein one or more of the hydrogen atoms are substituted with a heteroatom. Non limiting examples of heteroatoms are F, Cl, O and N. This definition includes the cases where the heteroatom forms multiple bonds with a C atom (double or triple bond). The term “alkyl substituted by a heteroatom-containing group” refers to an alkyl group, wherein one or more of the hydrogen atoms are substituted with a heteroatom-containing group.

The term “heteroatom-containing group” refers to a hydrocarbon molecule or molecular fragment in which one or more carbon and/or hydrogen atom is replaced with a heteroatom, typically with O or N. This definition includes the cases where the heteroatom forms multiple bonds with a C atom (double or triple bond).

The term “alkyl interrupted by a heteroatom” refers to an alkyl, wherein a heteroatom is inserted between a carbon-carbon bond. For example, in the case the heteroatom is oxygen, the alkyl interrupted by a heteroatom may also be referred to as an alkoxyalkyl group. The term “alkyl interrupted by a heteroatom-containing group” refers to an alkyl, wherein a heteroatom containing group is inserted between a carbon-carbon bond. The term “heteroatom-containing group” has the same meaning as above.

Non-limiting examples of the term “heterocycle containing from 4 to 8 atoms” comprises pyrrolidine, piperidine, piperazine, and morpholine.

Non-limiting examples of the term “pesticide” (which includes biopesticide) comprises fungicides, herbicides, insecticides, acaricides, algicides, molluscicides, miticides, nematicides, biocides and rodenticides. Specific examples of pesticides can be found in the book “The Pesticide Manual”, 18^{th} Edition, British Crop Protection Council 2018.

The term “nitrogen fertilizer stabilizer” as used herein refers to an agent that prevents or slow down kinetics of biodegradation of the fertilizer. Non-limiting examples are urease or nitrification inhibitors, such as NBPT (N-(n-butyl)thiophosphoric triamide), DCD (dicyandiamide) and NPPT (N-(n-propyl)thiophosphoric triamide). Nitrification inhibitors delay the bacterial oxidation of the ammonium ion in fertilizers by inhibiting the activity of Nitrosomonas bacteria in the soil, which transform ammonium into nitrite. Urease inhibitors inhibit the transformation of urea to ammonia and CO₂. Fertilizer containing fertilizer stabilizer is often referred to as slow- or controlled-release fertilizer or enhanced efficiency fertilizer (EEF). Non-limiting examples of nitrification inhibitors comprise DCD, DMPP (3,4-dimethylpyrazole phosphate), nitrapyrin (2-chloro-6-(trichloromethyl)pyridine, TU (thiourea), MT (1-mercapto-1,2,4-triazole), AM (2-amino-4-chloro-6-methyl pyrimidine, ASU (1-amide-2-thiourea), HPLC (1H-1,2,4-triazole), 3,4-dimethylpyrazole succinic (DMPSA). Non-limiting examples of urease inhibitors comprise NBPT, NPPT and CNPT (cylohexylphosphoric triamide).

The term “composition” as used herein refers to a mixture comprising at least the compound of the invention and another ingredient/compound. This mixture may be homogeneous (i.e. a solution) or heterogeneous (i.e. a dispersion, emulsion, suspension, suspoemulsion).

The term “room temperature” as used herein refers to a temperature of 20 to 30° C., typically to a temperature of 25° C.

The terms “(a)”, “(b)”, “(c)” and the like in the description and in the claims are used for distinguishing features and are not necessarily used for describing a sequential or chronological order.

Preferred embodiments according to the invention are defined hereinafter. The preferred embodiments are preferred alone or in combination. Further, it is to be understood that the following preferred embodiments refer to all aspects of the present invention, i.e. the compound, the method, the composition as well as the use of the compound.

In an embodiment, the invention relates to a compound of formula (I):

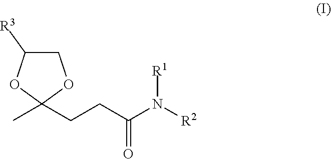

wherein
R¹ and R² are independently selected from —(C₁-C₆)alkyl including —(C₁)alkyl, —(C₂)alkyl, —(C₃)alkyl, —(C₄)alkyl, —(C₅)alkyl, and —(C₆)alkyl, optionally interrupted and/or substituted by a heteroatom or heteroatom-containing group, or R¹ and R² form with the nitrogen atom a heterocycle containing from 4 to 8 atoms; and
R³ is hydrogen, or —(C₁-C₄)alkyl, optionally interrupted and/or substituted by a heteroatom or heteroatom containing group;
with the proviso that when R¹ and R² are —CH₃, then R³ is not hydrogen.

In an embodiment, R¹ and R² are independently selected from —(C₁-C₃)alkyl including —(C₁)alkyl, —(C₂)alkyl, and —(C₃)alkyl. It is preferred that R¹ and R² are independently selected from methyl and ethyl, or form with the nitrogen atom a pyrrolidine, piperidine or a morpholine heterocycle. It is particularly preferred that R¹ and R² are both —CH₃.

In an embodiment, R³ is —(C₁-C₂)alkyl, optionally interrupted and/or substituted by a heteroatom, wherein the heteroatom is preferably oxygen. In an embodiment, R³ is hydrogen.

In a particular embodiment, notably when R¹ and R² are independently selected from —(C₁-C₃)alkyl including —(C₁)alkyl, —(C₂)alkyl, and —(C₃)alkyl, for instance when R¹ and R² are independently selected from methyl and ethyl, R³ is —(C₁-C₂)alkyl, i.e. R³ is —(C₁)alkyl or —(C₂)alkyl, preferably R³ is —CH₃.

In a preferred embodiment, R³ is —(C₁)alkyl (i.e. R³ is —CH₃) or hydroxymethyl (i.e. R³ is —CH₂—OH).

And thus, in a particularly preferred embodiment, the compound of formula (I) is 3-(2,4-dimethyl-1,3-dioxolan-2-yl)-N,N-dimethylpropanamide (i.e. R¹, R² and R³ are each —CH₃) or 3-(4-(hydroxymethyl)-2-methyl-1,3-dioxolan-2-yl)-N,N-dimethylpropanamide (i.e. R¹ and R² both —CH₃ and R³ is —CH₂—OH). Very preferably, the compound of formula (I) is 3-(2,4-dimethyl-1,3-dioxolan-2-yl)-N,N-dimethylpropanamide (i.e. R¹, R² and R³ are each —CH₃).

Advantageously, the compounds of formula (I) have good to excellent solubilization properties, are preferably biodedegradable and have preferably very good safety and sustainable ecotoxicity profiles, with none or very low hazard classification and none or very low ecotoxicity.

Preferably, the compounds of formula (I) have a renewable carbon index (RCI) of at least 0.80, in particular ranging from 0.80 to 1.

Renewable carbon commonly entails all carbon sources that avoid or substitute the use of any additional fossil carbon from the geosphere. Renewable carbon can come from the biosphere, atmosphere or technosphere, but not from the geosphere.

Renewable carbon is here defined as carbon derived from recently living plant or animal organisms (as opposed to carbon derived from fossil carbon which is coal, oil or petroleum based), as well as carbon derived from CO₂ capture.

Renewable Carbon Index (RCI) is defined as the value calculated by dividing the number of renewable carbons by the total number of carbons in the entire molecule. For example, if 80% of the number of carbons present is renewable carbon then the RCI is 0.8.

In an embodiment, the invention relates to a method for production of the compound of the present invention from levulinic acid or an ester or a salt thereof, comprising at least the following steps in any order:
(a) ketalization with a compound of formula R³—CH(OH)—CH₂OH
(b) amidification with an amine of formula HNR¹R²,
wherein R¹ to R³ are defined as above.

The ketalization in step (a) is preferably acid-catalyzed and is an equilibrium reaction generating water as a by-product. It is therefore preferably assisted by water removal during the reaction e.g. through distillation (for example vacuum distillation or azeotropic distillation using a so called Dean-Stark apparatus with the use of an appropriate solvent that forms an azeotrope with water such as toluene, xylene . . . ) in order to drive the equilibrium of the reaction toward completion. After the reaction, step a) preferably further comprises acid catalyst neutralization and removal.

In an embodiment, step (b) is base-catalyzed especially when starting from levulinic ester. In this case, an alcohol is generated as by-product. It is preferred that step (b) further comprises after reaction neutralization of the base catalyst and salt by-product removal. The base can preferably be neutralized, e.g. with an ion-exchange resin, such as amberlite which has (compared to a mineral acid) the advantage to avoid tedious salt by-product removal as the resin can be easily filtered off. Preferably, when neutralizing the base catalyst with an aqueous solution of an acid, the pH of the solution is controlled to avoid ketal hydrolysis under acidic conditions. When starting from levulinic acid, amidification in step b) does not require any catalyst and can be conducted thermally at temperatures in the range of 120-250° C. with removal of water. In that case an intermediate ammonium carboxylate salt is initially formed that is dehydrated to the amide by heating.

In a preferred embodiment, the production method starts from bio-based levulinic acid or an ester derivative thereof like methyl levulinate. The reaction

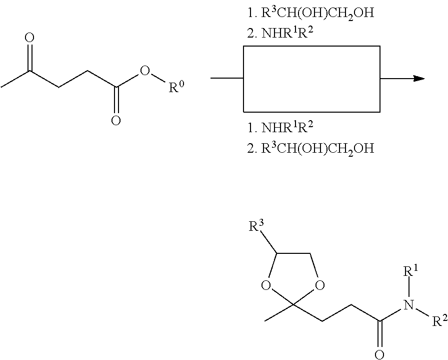

scheme is depicted below, wherein R⁰ is hydrogen or alkyl, preferably methyl or ethyl.

In a preferred embodiment, step (a) is performed first, followed by step (b). Both steps may comprise an additional purification step by the methods known in the art, such as (vacuum) distillation, filtration, liquid-liquid extraction, crystallisation or column chromatography.

Other methods for the production of the compound of the present invention may also be used. For instance, instead of starting from levulinic acid or an ester or a salt thereof, one may start directly from ketal-ester intermediates like those obtained in step (a), and only perform step (b) of the above described method. As a non-limitative example, 3-(4-(hydroxymethyl)-2-methyl-1,3-dioxolan-2-yl)-N,N-dimethylpropanamide can be obtained, through step (b), from ethyl glycerol-ketal levulinate which is prepared following the protocol described in example 6 of WO 2012/018939, by using methanesulfonic acid as catalyst instead of 2-Naphtalenesulfonic acid. And the other way around: one may start directly from amine intermediates like those obtained in step (b) and only perform step (a) of the above described method.

In an embodiment, the present invention relates to a composition comprising the compound of the present invention or a mixture of compounds of the present invention. This composition may be at least part of a formulation aimed at a specific function, for instance a phytosanitary (or agriculture) formulation, a cleaning formulation, a stripping formulation, a degreasing formulation, a lubricant or textile formulation, a coating formulation, for example a paint formulation or a pigment or ink formulation.

In a very advantageous embodiment, the composition according to the invention is a phytosanitary (or agriculture) formulation, preferably a concentrated phytosanitary (or agriculture) formulation, i.e. a formulation comprising a phytosanitary (agricultural) active compound. Agriculture uses many active substances such as fertilizers, biostimulants, plant growth regulators, natural plant defense enhancers, inoculants or pesticides (for example herbicides, insecticides, acaricides, fungicides, algicides, molluscicides, miticides, nematicides, biocides, or rodenticides, for instance a raticide).

This phytosanitary (or agriculture) formulation may comprise:
a) a phytosanitary (agricultural) active compound;
b) the compound of the invention, generally as solvent;
c) optionally at least one emulsifier, preferably a surfactant; and
d) optionally water.

As used herein, the terms “phytosanitary active compound” or “agricultural active compound” mean an active ingredient used in particular to the practice of farming, including cultivation of the soil for the growing of crops. However, the use of agricultural (phytosanitary) active compound is not limited to application to crops. Agricultural active compounds (or materials) may be applied to any surface, e.g., for the purpose of cleaning or aiding or inhibiting growth of a living organism. Other non-crop applications include, but are not limited to, application to turf and ornamentals, and application to railroad weed.

The agricultural (phytosanitary) active compounds are generally products in pure or highly concentrated form.

The agricultural (or phytosanitary) active compounds are preferably chosen from pesticides (including biopesticides), fertilizers, fertilizer stabilizers, nutrients, biostimulants, plant growth regulators, natural plant defense enhancers, inoculants and mixtures thereof.

Advantageously, the phytosanitary (or agriculture) formulation according to the invention comprises at least one pesticide.

For example, the at least one pesticide may be chosen from fungicides, herbicides, insecticides, acaricides, algicides, molluscicides, miticides, nematicides, biocides and rodenticides (for example raticides).

Non-limiting examples of fungicides suitable for use in the agriculture (or phytonanitary) formulation of the present invention include azoles such as e.g. prothioconazole, epoxiconazole, difenoconazole, propiconazole, cyproconazole, tebuconazole; strobilurins such as e.g. azoxystrobin, trifloxystrobin, picoxystrobin, fluoxastrobin, pyraclostrobin; and SDHIs (carboxamides) such as bixafen, fluxapyroxad, benzovindiflupyr, fluopyram; and mixtures thereof.

The agricultural (phytosanitary) active compounds can be water-insoluble, at 20° C. and at atmospheric pressure (i.e., 1.013×10⁵ Pa).

In particular, the agricultural (phytosanitary) active compounds can be soluble in water to no more than 100 g/L, generally no more than 20 g/L, notably no more than 5 g/L, for instance no more than 1 g/L and even no more than 0.2 g/L, at 20° C. and at atmospheric pressure (i.e., 1.013×10⁵ Pa).

In a further embodiment, the compound or composition of the present invention forms part of a fertilizer formulation, preferably an enhanced efficiency fertilizer formulation, which comprises a fertilizer and/or a fertilizer stabilizer, in particular a nitrogen fertilizer and/or a nitrogen fertilizer stabilizer and/or a urease and/or nitrification inhibitor.

The fertilizer and/or a fertilizer stabilizer, in particular the nitrogen fertilizer and/or nitrogen fertilizer stabilizer and/or urease and/or nitrification inhibitor may be N-(n-butyl)thiophosphoric acid triamide (NBPT) and/or dicyandiamide (DCD).

In another embodiment, said fertilizer formulation further comprises at least one biostimulant, one plant growth regulator, one natural plant defense enhancer and/or one inoculant.

In another embodiment, said fertilizer formulation further comprises at least one pesticide, for example a fungicide, an herbicide, an insecticide, an acaricide, an algicide, a molluscicide, a miticide, a nematicide, a biocide or a rodenticide (for example a raticide).

Generally, the amount of phytosanitary (agricultural) active compound(s) in the phytosanitary (agriculture) formulation of the invention ranges from 0.01 to 90% by weight, preferentially from 0.1 to 90% by weight more preferentially from 0.1 to 80% by weight; even more preferentially from 0.5 to 70% by weight; better from 1 to 65% by weight, in particular from 5 to 60% by weight, and for instance from 10 to 60% by weight, relative to the total weight of the phytosanitary (agriculture) formulation.

According to a particular embodiment of the invention (concentrated composition), the total content of agricultural (phytosanitary) active compound(s) in the agriculture (phytosanitary) formulation ranges from 5 to 90% by weight, preferentially from 5 to 70% by weight, more preferentially from 5 to 60% by weight, and in particular from 10 to 60% by weight, relative to the total weight of the agriculture (phytosanitary) formulation.

According to another particular embodiment of the invention (diluted composition), the total content of agricultural (phytosanitary) active compound(s) in the agriculture (phytosanitary) formulation ranges from 0.01 to 3% by weight, preferentially from 0.05 to 2% by weight, and more preferentially from 0.1 to 1% by weight, relative to the total weight of the agriculture (phytosanitary) formulation.

Generally, the compound of the invention or the mixture of compounds of the invention represents from 10 to 99.9% by weight, preferentially from 10 to 99% by weight, more preferentially from 20% to 95% by weight, in particular from 30% to 90% by weight, for instance from 30% to 80% by weight, relative to the total weight of the phytosanitary (agrochemical) formulation.

In an embodiment, the phytosanitary (agriculture) formulation according to the invention further comprises a fertilizer and/or a fertilizer stabilizer, in particular a nitrogen fertilizer and/or a nitrogen fertilizer stabilizer. The fertilizer stabilizer may be N-(n-butyl)thiophosphoric acid triamide (NBPT) and/or dicyandiamide (DCD).

In another embodiment, said phytosanitary formulation further comprises at least one biostimulant, one plant growth regulator, one natural plant defense enhancer and/or one inoculant.

The compounds according to the present invention advantageously show good solubilization properties.

The present invention also relates to the use of the compound of the present invention as a solvent, co-solvent and/or crystallization inhibitor, in particular in agriculture (phytosanitary) formulations comprising an active ingredient (in particular a pesticide) and fertilizer formulations comprising a fertilizer and/or a fertilizer stabilizer, in particular a nitrogen fertilizer and/or a nitrogen fertilizer stabilizer and/or a urease and/or nitrification inhibitor.

The compound of the present invention may also be used as a coalescing agent, for instance in aqueous paint formulations.

The compound of the invention may be used in an agriculture formulation comprising an active ingredient, for instance a pesticide.

The compound of the present invention may also be used in a fertilizer formulation, preferably an enhanced efficiency fertilizer formulation, comprising a fertilizer and/or a fertilizer stabilizer, in particular a nitrogen fertilizer and/or a nitrogen fertilizer stabilizer and/or a urease and/or nitrification inhibitor.

The term solvent may especially denote a product that is liquid at the usage temperature, which may contribute to rendering a solid substance liquid, or to preventing/retarding the solidification or crystallisation of material in a liquid form. It may generally have a melting point less than or equal to 20° C., in particular 5° C., for example 0° C.

It is possible to combine several agricultural (phytosanitary) active compounds in the agriculture (phytosanitary) formulation of the invention comprising the compound of the invention or a mixture of compounds of the invention.

The compound of the present invention may also be used generally as a replacement for potentially toxic solvents such as DMSO (dimethylsulfoxide), NMP (N-methyl 2-pyrrolidone), DMF (N,N-dimethyl formamide), DMAc (N,N-dimethyl acetamide), in particular to dissolve polymers, or as a replacement for other polar and eco-friendly solvents, such as NBP (N-butyl-2-pyrrolidone), Cyrene® (Dihydrolevoglucosenone), Rhodiasolv® PolarClean, N,N-dimethyl lactamide and Rhodiasolv® ADMA 10.

The agriculture (phytosanitary) formulation according to the invention may optionally comprise at least one biostimulant.

The term “biostimulant” is preferably intended to mean a compound which may enhance metabolic or physiological processes such as respiration, photosynthesis, nucleic acid uptake, ion uptake, nutrient delivery, or a combination thereof.

Generally, this is a substance or microorganism that, when applied to seeds, plants or on the rhizosphere, can stimulate natural processes to enhance or benefit nutrient uptake, nutrient use efficiency, tolerance to abiotic stress, or crop quality and yield.

Non-limiting examples of biostimulants include seaweed extracts (e.g., ascophyllum nodosum), humic acids (e.g., potassium humate), fulvic acids, myoinositol, glycine, and combinations thereof.

The agricultural (phytosanitary) formulation according to the invention may optionally comprise at least one plant growth regulator.

Plant growth regulators mean active ingredients used to influence the growth characteristics of plants. Examples of plant growth regulators which may be used in the present invention include, but are not limited to: 1-naphthaleneacetic acid, 1-naphthaleneacetic acid -salt, 1-napthol, 2,4-dichlorophenoxyacetic acid (2,4-D), 2,4-DB, 2,4-DEP, 2,3,5-triiodobenzoic acid, 2,4,5-trichlorophenoxyacetic acid, 2-naphthoxyacetic acid, 2-naphthoxyacetic acid sodium salt, 3-chloro-4-hydroxyphenylacetic acid, 3-indoleacetic acid, 4-biphenylacetic acid, 4-chlorophenoxyacetic acid (4-CPA), 4-hydroxyphenylacetic acid, 6-benzylaminopurine, auxindole, a-naphthaleneacetic acid K-salt, β-naphfhoxyacetic acid, p-chlorophenoxyacetic acid, dicamba, dichlorprop, fenoprop, indole-3-acetic acid (IAA), indole-3-acetyl-DL-aspartic acid, indole-3-acetyl-DL-tryptophan, indole-3-acetyl-L-alanine, indole-3-acetyl-L-valine, indole-3-butyric acid (IBA), indole-3-butyric acid K-salt, indole-3-propionic acid; a-naphthaleneacetic acid, methyl indole-3-acetate, naphthaleneacetamide, naphthaleneacetic acid (NAA), phenylacetic acid, picloram, potassium naphthenate, sodium naphthenate, 4-hydroxyphenethyl alcohol, 4-CPPU, 6-benzylaminopurine (BA), 6-(Y,Y-dimethylallylamino)purine (2iP), 2-iP-2HC1, adenine, adenine hemisulfate, benzyladenine, kinetin, meta-topolin, N6-benzoyladenine, N-benzyl-9-(2-tetrahydropyranyl) adenine (BP A), N-(2-chloro-4-pyridyl)-N-phenylurea, gibberellic acid (GA3), gibberellins, gibberellins A4+A7 (GA n), ethylene and abscisic acid.

The agriculture formulation (or agrochemical formulation) according to the invention may optionally comprise at least one emulsifier.

Emulsifiers are agents that are intended to facilitate emulsification after the formulation is placed in the presence of water, and/or stabilisation (over time and/or in temperature) of the emulsion, for example by avoiding separation of the phases.

Generally, the total amount of emulsifier(s) in the agriculture (phytosanitary) formulation according to the invention, ranges from 0.05 to 40% by weight, preferentially from 0.1 to 35% by weight, more preferentially from 0.5 to 30% by weight, in particular from 1 to 25% by weight, for instance from 1 to 5% by weight, relative to the total weight of the agriculture (phytosanitary) formulation.

Generally, the agrochemical (phytosanitary) formulation according to the invention may further at least one surfactant.

Advantageously, the surfactants that may be used in the invention are chosen from anionic, non-ionic, cationic, amphoteric or zwitterionic surfactants, and mixtures thereof.

Preferentially, the surfactants are chosen from anionic surfactants, non-ionic surfactants, and mixtures thereof.

More preferentially, the surfactants are chosen from anionic surfactants, polyalkoxylated non-ionic surfactants, and mixtures thereof.

The emulsifiers and surfactants that may be used are different from the agricultural active compound(s).

By way of examples of anionic surfactants, mention may be made without any intended limitation thereto, of:
alkylsulfonic acids, arylsulfonic acids, optionally substituted with one or more hydrocarbon groups, and the acid function of which is partly or totally salified, like C8-C50 alkylsulfonic acids, more particularly C8-C30, preferably C10-C22 alkylsulfonic acids, benzenesulfonic acids, naphthalenesulfonic acids, substituted with one to three C1-C30, preferably C4-C16 alkyl and/or C2-C30, preferably C4-C16 alkenyl groups,
mono- or di-esters of alkylsulfosuccinic acids, of which the linear or branched alkyl portion is optionally substituted with one or more linear or branched C2-C4 hydroxylated and/or alkoxylated (preferably ethoxylated, propyxylated, ethopropoxylated) groups,
phosphate esters more particularly selected from among those comprising at least one linear or branched, saturated, unsaturated or aromatic hydrocarbon group, comprising 8 to 40 carbon atoms, preferably 10 to 30, optionally substituted with at least one alkoxylated (ethoxylated, propoxylated, ethopropoxylated) group. In addition, they comprise at least one phosphate ester group, mono- or di-esterified such that it is possible to have one or two free or partly or totally salified groups. The preferred phosphate esters are of the type of the mono- and di-esters of phosphoric acid and of alkoxylated (ethoxylated and/or propoxylated) mono-, di- or tri-styrylphenol, or alkoxylated (ethoxylated and/or propoxylated) mono-, di- or trialkylphenol, optionally substituted with one to four alkyl groups; of phosphoric acid and of an alkoxylated (ethoxylated or ethopropoxylated) C8-C30, preferably C10-C22 alcohol; of phosphoric acid and of a non-alkoxylated C8-C22, preferably C10-C22 alcohol,
sulfate esters obtained from saturated or aromatic alcohols optionally substituted with one or more alkoxylated (ethoxylated, propoxylated, ethopropoxylated) groups, and for which the sulfate functions appear in the free acid form, or are partly or totally neutralised. As an example, mention may be made of sulfate esters more particularly obtained from saturated or unsaturated C8-C20 alcohols, which may comprise 1 to 8 alkoxylated (ethoxylated, propoxylated, ethopropoxylated) units; sulfate esters obtained from polyalkoxylated phenol, substituted with 1 to 3 saturated or unsaturated C2-C30 hydroxycarbon groups, and in which the number of alkoxylated units is comprised between 2 and 40; the sulfate esters obtained from polyalkoxylated mono-, di- or tri-styrylphenol in which the number of alkoxylated units varies from 2 to 40.

The anionic surfactants may be in the acid form (they are potentially anionic), or in a partly or totally salified form with one counter-ion. The counter-ion may be an alkali metal, such as sodium or potassium, an alkaline earth metal, such as calcium, or moreover even an ammonium ion of formula N(R)4+ in which the R groups, either identical or different, represent a hydrogen atom or a C1-C4 alkyl group optionally substituted with an oxygen atom.

By way of examples of non-ionic surfactants, mention may be made without any intended limitation thereto, of:
polyalkoxylated (ethoxylated, propoxylated, ethopropoxylated) phenols substituted with at least one C4-C20, preferably C4-C12 alkyl group, or substituted with at least one alkylaryl group, the alkyl portion of which is a C1-C6 alkyl. More particularly, the total number of alkloxylated units is comprised between 2 and 100. As an example, mention may be made of polyalkoxylated mono-, di- or tri-(phenylethyl) phenols, or polyalkoxylated nonylphenols. Amongst the ethoxylated and/or propoxylated, sulfated and/or phosphated di- or tri-styrylphenols, mention may be made of ethoxylated di-(phenyl-1-ethyl)phenol, containing 10 oxyethylene units; ethoxylated di-(phenyl-1-ethyl)phenol, containing 7 oxyethylene units; sulfated ethoxylated di-(phenyl-1-ethyl)phenol, containing 7 oxyethylene units; ethoxylated tri-(phenyl-1-ethyl)phenol, containing 8 oxyethylene units; ethoxylated tri-(phenyl-1-ethyl)phenol, containing 16 oxyethylene units; sulfated ethoxylated tri-(phenyl-1-ethyl)phenol, containing 16 oxyethylene units; ethoxylated tri-(phenyl-1-ethyl)phenol, containing 20 oxyethylene units; phosphated ethoxylated tri-(phenyl-1-ethyl) phenol, containing 16 oxyethylene units.
polyalkoxylated (ethoxylated, propyxylated, ethopropoxylated) C6-C22 fatty acids or alcohols. The number of alkoxylated units is comprised between 1 and 60. The term ethoxylated fatty acid includes both the products obtained by ethoxylation of a fatty acid by ethylene oxide as well as those obtained by esterification of a fatty acid by a polyethylene glycol.
polyalkoxylated (ethoxylated, propoxylated, ethopropoxylated) triglycerides of vegetable or animal origin. Thus, may be included triglycerides from lard, tallow, ground nut oil, butter oil, cotton seed oil, flax oil, olive oil, palm oil, grapeseed oil, fish oil, soya bean oil, castor oil, rapeseed oil, coprah oil, coconut oil, and comprising a total number of alkoxylated units comprised between 1 and 60. The term ethoxylated triglyceride makes reference both to products obtained by ethoxylation of a triglyceride with ethylene oxide as well as to those obtained by transesterification of a triglyceride with a polyethylene glycol.
sorbitan esters, optionally polyalkoxylated (ethoxylated, propoxylated, ethopropoxylated), more particularly the cyclised sorbitol esters of C10-C20 fatty acids such as lauric acid, stearic acid, or oleic acid, and comprising a total number of alkoxylated units comprised between 2 and 50.

Useful emulsifiers are in particular the following products, all marketed by the Applicant:
Soprophor® TSP/724: a surfactant based on ethopropoxylated tristyrylphenol,
Soprophor® 796/P: a surfactant based on ethopropoxylated tristyrylphenol
Soprophor® CY 8: a surfactant based on ethoxylated tristyrylphenol
Soprophor® BSU: a surfactant based on ethoxylated tristyrylphenol
Soprophor® S/25: a surfactant based on ethoxylated tristyrylphenol
Soprophor® 3D33: a surfactant based on ethoxylated tristyrylphenol phosphate
Alkamuls® RC: a surfactant based on ethoxylated castor oil
Alkamuls® OR/36: a surfactant based on ethoxylated castor oil
Alkamuls® V02003: a surfactant based on ethoxylated castor oil
Alkamuls® OL40: a surfactant based on ethoxylated sorbitan hexaoleate
Alkamuls® T/20: a surfactant based on ethoxylated sorbitan ester.
Geronol® TBE724: a surfactant based on ethopropoxylated tristyrylphenol
Geronol® TEB25: a mixture of surfactants based on ethoxylated castor oil, calcium dodecyl benzene sulfonate and alkoxylated polymers
Rhodacal® 60/B: a surfactant based on dodecylbenzene sulphonate
Rhodacal® 60/BE: a surfactant based on dodecylbenzene sulphonate.

Generally, the total amount of surfactant(s) in the agriculture (phytosanitary) formulation according to the invention, ranges from 0.05 to 40% by weight, preferentially from 0.1 to 35% by weight, more preferentially from 0.5 to 30% by weight, in particular from 1 to 25% by weight, for instance from 1 to 5% by weight, relative to the total weight of the agriculture (phytosanitary) formulation.

Generally, the total amount of anionic surfactant(s) in the agriculture (phytosanitary) formulation according to the invention, ranges from 0.05 to 40% by weight, preferentially from 0.1 to 35% by weight, more preferentially from 0.5 to 30% by weight, in particular from 1 to 25% by weight, for instance from 1 to 5% by weight, relative to the total weight of the agriculture (phytosanitary) formulation.

Generally, the total amount of non-ionic surfactant(s), in particular polyalkoxylated non-ionic surfactant(s) in the agriculture (phytosanitary) formulation according to the invention, ranges from 0.05 to 40% by weight, preferentially from 0.1 to 35% by weight, more preferentially from 0.5 to 30% by weight, in particular from 1 to 25% by weight, for instance from 1 to 5% by weight, relative to the total weight of the agriculture (phytosanitary) formulation.

The phytosanitary (agriculture) formulation according to the invention may optionally further comprise at least one co-solvent, different from the compound of the invention.

This other solvent or co-solvent is generally selected from:
linear or branched, saturated or unsaturated, aliphatic hydrocarbons, possibly containing a halogen-, phosphorus-, sulfur—and/or nitrogen atom and/or a functional group,
carbocyclic or heterocyclic hydrocarbons, whether saturated, unsaturated or aromatic, possibly containing a halogen-, phosphorus-, sulfur—and/or nitrogen atom and/or a functional group, More particularly, this co-solvent is chosen from:
alkanes, cycloalkanes and aromatic derivatives, for example paraffins with a branched chain or straight chain such as “white oil” or decalin; mono-, di- or tri alkyl benzenes or naphthalenes, the compounds sold under the name Solvesso® 100, 150, 200 standard and ND grades;
aliphatic, cycloaliphatic or aromatic mono-, di- or tri-esters, for example alkyl alkanoates such as methyl oleate; benzyl alkanoates; alkyl benzoates;
gamma butyrolactone; caprolactone; esters of glycerol and citric acid; alkyl salicylates; phthalates; dibenzoates; acetoacetates; glycol ether acetates, dipropylene glycol diacetate;
alkyl mono-, di-, or tri-phosphates such as for example triethyl phosphate; tributyl phosphate; or tri-2-ethylhexylphosphate;
aliphatic, cycloaliphatic or aromatic ketones such as for example dialkyl ketones; benzyl ketones; fenchone; actetophenone; cyclohexanone; alkyl cyclohexanones;
aliphatic, cycloaliphatic or aromatic alcohols such as for example glycols; 2-ethylhexanol; cyclohexanol; benzyl alcohols; tetrahydrofurfuryl alcohol;
aliphatic, cycloaliphatic or aromatic ethers such as for example ethers of glycol, notably ethylene and propylene glycol, and their polymers; diphenyl ether, dipropylene glycol; monomethyl or monobutyl ether, monobutyl ether of tripropylene glycol; alkoxyalkanols; dimethyl isosorbide;
fatty acids such as for example linoleic acid, linolenic acid, oleic acid;
carbonates such as for example propylene or butylene carbonate; lactates; fumarates, succinates, adipates, maleates;
amides such as for example alkyldimethylamides, dimethyl-decanoamide;
alkyl ureas;
amines such as for example alkanolamines, morpholine; N-alkyl-pyrrolidones;
tetramethyl sulfone;
dimethyl sulfoxide;
halogenoalkanes or halogenated aromatic solvents such as for example chloroalkanes or chlorobenzene.

Crystallisation inhibitors may also be present in the phytosanitary (agriculture) formulations according to the invention. Crystallisation inhibitors may be the co-solvents mentioned here above. Crystallisation inhibitors may also be non-polyalkoxylated fatty alcohols or fatty acids, for example mention may be made of the product Alkamuls® OL700 marketed by the Applicant, alkanolamides, polymers.

The agriculture (phytosanitary) formulation according to the invention may further contain one or more additives different from the ingredients described previously, and which are preferably chosen from viscosity modifying agents, suspending agents, antifoam agents and defoamers, in particular silicone antifoams and defoamers, anti-rebound agents, anti-leaching agents, penetration adjuvants, inert fillers, in particular mineral fillers, binders, diluents, anti-freeze agents, stabilisers, dyes, emetic agents, stickers (adhesion promoters), absorbents, dispersants, disintegration agents, wetting agents, preservatives and/or anti-microbials.

Each additive can be present in the agriculture (phytosanitary) formulation according to the invention in an amount ranging from 0 to 20% by weight, preferably from 0 to 10% by weight, relative to the total weight of the agriculture formulation. Each additive can be for instance present in the agricultural (phytosanitary) formulation according to the invention in an amount ranging from 0.1 to 20% by weight, in particular from 0.1 to 10% by weight, relative to the total weight of the formulation. Each additive can be present in the agrochemical (phytosanitary) formulation according to the invention in an amount preferably ranging from 0 to 5% by weight, notably from 0.1 to 5% by weight, relative to the total weight of the formulation. A person skilled in the art will be able to choose these optional additives and their amounts so that they do not harm the properties of the agriculture (phytosanitary) formulation of the present invention.

Advantageously, the agriculture (phytosanitary) formulation according to the invention is in a liquid form, at 20° C. and at atmospheric pressure (i.e., 1.013×105 Pa) and may be in the form of a concentrate of agricultural (phytosanitary) active compound(s), a diluted concentrate, or a sprayable diluted.

Different types of formulation may be used according to the different agricultural (phytosanitary) active compound(s). The formulations that it is possible to use depend on the physical form of the agricultural (phytosanitary) active materials (for example solid or liquid) and on their physicochemical properties in the presence of other compounds such as water or solvents.

For practical reasons (for example for reasons of ease of handling), it may be preferred to use formulations in liquid form. Depending on the physicochemical properties of the different agricultural (phytosanitary) active compound(s) considered, formulations can be in the form of emulsifiable concentrates (EC), concentrated emulsions in water (EW), microemulsions (ME), suspoemulsions (SE), oil dispersions (OD), dispersible concentrates (DC), suspension concentrates (SC), capsule suspensions (CS), soluble liquids (SL), flowable concentrates for seed treatments (FS).

Preferably, the agriculture (phytosanitary) formulation according to the invention is in the form of an emulsifiable concentrate (EC), concentrated emulsion in water (EW), microemulsion (ME), suspoemulsion (SE), oil dispersion (OD), dispersible concentrate (DC), capsule suspension (CS), soluble liquid (SL).

More preferentially, the agriculture (phytosanitary) formulation according to the invention is in the form of an emulsifiable concentrate, an emulsion in water concentrate, a microemulsion concentrate, a suspoemulsion concentrate, an oil dispersion concentrate or a dispersible concentrate.

In a particular embodiment, the agriculture (phytosanitary) formulation according to the invention is in the form of an emulsifiable concentrate (EC).

The agriculture (phytosanitary) formulation according to the invention is generally a concentrated agrochemical (phytosanitary) formulation and is intended to be spread out over a cultivated field or a field to be cultivated, most often after dilution with water, in order to obtain a diluted composition. Dilution is generally carried out by the farm operator, directly in a tank (“tank-mix”), for example in the tank of a device intended to spread out the composition. This does not exclude the possibility of the farm operator adding other plant-protective products, for example fungicides, herbicides, pesticides, insecticides, fertilisers, adjuvants, etc. Thus, the formulation may be used for preparing a formulation diluted in water of the agricultural (phytosanitary) active compound(s), by mixing at least one part by weight of concentrated formulation with at least 10 parts of water, preferably less than 10,000 parts. The dilution ratios and the amounts to be applied over the field generally depend on the agricultural (phytosanitary) active compound(s) and on the desirable dose for treating the field (this may be determined by the farm operator).

According to one embodiment of the invention, the agrochemical (phytosanitary) formulation according to the invention is aqueous.

According to this embodiment, the water content of the agriculture (phytosanitary) formulation preferably ranges from 5 to 99% by weight, more preferentially from 20 to 95% by weight, even more preferentially from 25 to 90% by weight, in particular from 25 to 85% by weight, for instance from 25 to 70% by weight, relative to the total weight of the agriculture (phytosanitary) formulation.

According to this embodiment, the pH preferably ranges from 1 to 11, and particularly from 2.5 to 9.5.

The pH of the formulations can be adjusted to the desired value by means of basifying agents or acidifying agents. Use may be made, among the basifying agents, of one or more alkaline agents, such as ammonia, sodium hydroxide or ethanolamine. Mention may be made, by way of examples, among the acidifying agents, of inorganic or organic acids, such as hydrochloric acid or orthophosphoric acid.

According to a particular embodiment of the invention, the phytosanitary (agriculture) formulation may advantageously comprise:
a) from 0.01 to 90% by weight, preferably from 5 to 60% by weight, of at least one agricultural (phytosanitary) active compound (only one agricultural (phytosanitary) active compound or a combination of different agricultural (phytosanitary) active compounds), preferably at least one pesticide, relative to the total weight of the phytosanitary (agriculture) formulation,
b) from 5 to 90% by weight, preferably from 10 to 90% by weight, in particular from 30 to 90% by weight, for instance from 30 to 80% by weight, of a compound of the invention or of a mixture of compounds according to the present invention, relative to the total weight of the phytosanitary (agriculture) formulation,
c) optionally, from 0.1 to 40% by weight, preferably from 1 to 30% by weight, of at least one said co-solvent, relative to the total weight of the phtosanitary (agriculture) formulation,
d) from 0.05 to 40% by weight, preferably from 0.1 to 35% by weight, more preferentially from 0.5 to 30% by weight, in particular from 1 to 25% by weight, for instance from 1 to 5% by weight, of at least one surfactant, relative to the total weight of the phytosanitary (agriculture) formulation,
e) optionally, from 5 to 90% by weight, preferably from 10 to 80% by weight, in particular from 25 to 70% by weight, of water, relative to the total weight of the phytosanitary (agriculture) formulation.

Known conventional methods for preparing agriculture (phytosanitary) formulations may be implemented. It is possible to undertake this by simply mixing the constituents.

The agriculture (phytosanitary) formulation according to the invention may be used to kill or inhibit pests and/or clean and/or inhibit growth of undesired plants.

The agriculture (phytosanitary) formulation according to the invention can be diluted and applied to at least one plant, area adjacent to a plant, soil adapted to support growth of a plant, root of a plant, foliage of a plant, and/or seed adapted to produce a plant, in a customary manner; for example by watering (drenching), drip irrigation, spraying, and/or atomizing.

In the above description, all the preferred embodiments with regard to the components may be used individually or in combination.

As mentioned above, the compound of the present invention may also be used generally as a replacement for polar solvents, such as NMP, DMF, DMSO, acetophenone and DMAc, in particular to dissolve polymers. For example, these solvents can be used during wet coating of electrodes, such as electrodes for car batteries, with a polymer binder, such as PVDF (polyvinylidene fluoride), and a carbon material. The compound of the present invention is also useful for other coating applications, the manufacturing of membranes, or solid batteries. It can also be used as a solvent in recycling processes of polymers, especially chemically resistant polymers like PVDF or PVDC (polyvinylidene chloride), still as a replacement of the above mentioned solvents. It can also been used for the preparation, in solution, of polycondensates, especially polyimides or polyesters or polyamides or polyamide-imides, especially partially or completely aromatic polycondensates such as aromatic polyamides (aramids). Finally, it can also be used as cleaning solvent for the cleaning of equipments like reactors for instance, in particular polymerization reactors.

Since the compounds of the invention are advantageously eco-friendly solvents and have preferably very good safety and suustainable profiles, they can also be used as solvents in household care formulations, used in homes or in public areas (hotels, offices, factories, etc.) They may be formulations for cleaning hard surfaces such as floors, the surfaces of furniture and of kitchen and bathroom fittings, or dishes. These formulations may also be used in the industrial sphere for degreasing manufactured products and/or for cleaning them.

### EXAMPLES

### Example 1

### Synthesis of 3-(2,4-dimethyl-1,3-dioxolan-2-yl)-N,N-dimethylpropanamide

### Ketalization of methyl levulinate with 1,2-propylene glycol

In a 1 L double-jacketed reactor equipped with a temperature probe, a mechanical stirrer (propeller with 3 inclined plows) and baffles, a cooler (5° C.) and a Dean-Stark apparatus, were added at room temperature:
500 mL of toluene.
150.47 g of methyl levulinate (1.16 moles, 1 eq.).
218.38 g of 1,2-propylene glycol (2.87 moles, 2.5 eq.).

The reaction medium (biphasic at room temperature) was then allowed to stir at 515 rpm and the temperature of the reaction mass was then increased to 107° C. At this temperature, the solution turned homogeneous and transparent.

The catalyst, methanesulfonic acid (1.5 mL, 2.22 g, 23.05 mmoles, 2 mol %) was then added in one shot into the solution causing an important refluxing of the reaction mass. Following the introduction of the catalyst, water was formed and its condensation was observed into the Dean-Stark apparatus.

The reaction mixture was then allowed to stir at reflux (109-110° C.) during 3 h15 until there was no more water condensing into the Dean-Stark. At this stage of the reaction, ¹H NMR analysis of the crude shows that 75% of ketones was converted to ketal and that there was significant formation of trans-esterified by-products with 1,2-propylene glycol. The reaction mixture was then allowed to cool down at room temperature and the catalyst was neutralized through the addition of 200 mL of a saturated aqueous NaHCO₃ solution.

The phases were decanted and separated and the organic phase was washed 4 times with 200 mL of a saturated NaHSO₃ aqueous solution in order to remove selectively ketone products from the organic phase through the reversible and selective formation of α-hydroxysulfonate derivatives which were water soluble.

Finally, the organic phase was washed with 200 mL of brine and 200 mL of water.

The organic phase was then dried over MgSO₄, filtered and the solvent was removed under vacuum to afford 144.7 g of crude material as an oil.

¹H NMR analysis of the crude showed that the product contained around 96 mol % of ketal (with 4 mol % of ketone) and that 44 mol % of the ketal was trans-esterified with 1,2-propylene glycol. Therefore, in order to convert back the trans-esterified by-product to the desired methyl ester the crude was directly engaged in a base catalyzed transesterification reaction in MeOH.

The trans-esterification was carried out as follows:

In a 1 L double-jacketed reactor equipped with a temperature probe, a mechanical stirrer (propeller with 3 inclined plows), baffles and a cooler (5° C.) were added at room temperature:
300 mL of methanol,
144.66 g of crude material from previous step,
1.158 g of sodium methylate (0.8 wt % wrt to crude).

The mixture was then allowed to stir at 50° C. (500 rpm) during 15 minutes allowing the reaction to proceed to completion according to ¹H NMR analysis.

The reaction mass was then cooled down at room temperature and 0.5 mL of an aqueous H₃PO₄ solution (85 wt %) was added to the mixture in order to decrease down the pH between 7.5 and 8 (in this case 7.3 after 10% dilution in water at room temperature).

Methanol was removed under vacuum and the crude was washed with 100 mL of a saturated NaHCO₃ aqueous solution, 100 mL of a saturated Na₂SO₃ aqueous solution and finally 100 mL of water.

The crude was dried under vacuum (80° C., 30 mbar) in order to recover 104.65 g of the desired methyl ketal levulinate.

¹H NMR analysis showed that the crude contained only 2 mol % of methyl levulinate and 98 mol % of ketal and was allowed to be directly engaged in the next step;

¹H NMR (CDCl₃, 400 MHz) δ (ppm): 4.2-4.0 (m, 1H), 3.98-3.86 (m, 1H), 3.56 (s, 3H), 3.33 (t), 3.25 (t) (sum of integrals: 1H), 2.38-2.20 (m, 2H), 1.98-1.82 (m, 2H), 1.25 (s), 1.21 (s) (sum of integral: 2H), 1.15 (d, 3H).

¹³C (CDCl₃, 101 MHz) δ (ppm): 173.22, 173.14, 108.85, 108.72, 72.26, 71.30, 70.84, 70.47, 50.82, 50.77, 34.18, 33.89, 28.50, 28.06, 24.67, 23.84, 18.19, 17.38.

The ester functional group of methyl 3-(2,4-dimethyl-1,3-dioxolan-2-yl)propanoate was then converted into the amide in carefully dried vessels and under an inert argon atmosphere as follows: In a dried 1 L double-jacketed reactor equipped with a mechanical stirrer (propeller with 3 inclined plows), baffles, a temperature probe and a cooler (5° C.) were added at room temperature:
103.08 g of methyl ketal levulinate obtained in the previous step (assumed 100% purity, 0.55 mole),
582 mL of DMA/MeOH solution (427.90 g, 2M, 11 wt %, 1.04 moles, 1.9 eq.).

The solution was then allowed to stir (600 rpm) at room temperature and 0.914 g of solid NaOMe (0.017 mole, 3 mol %) was added into the solution. The solution was then allowed to stir at 50° C. during 7 h00 and an additional amount of sodium methylate was added into the solution (2.08 g, 7 mol %). The mixture was then stirred at 50° C. during 62 h00. At this stage, the conversion of methyl levulinate to the desired N,N-dimethylamide reached >98%.

The volatiles (DMA and MeOH) were then removed under vacuum (65° C., 400 mbar) and the catalyst in the residue was carefully neutralized thanks to the addition at room temperature of 3.8 g of aqueous H₃PO₄ solution (85 wt %), followed by 3 mL of aqueous NaOH solution (30 wt %) in order to decrease down the pH of the mixture between 7.5 and 8 (final pH=7.4, measured after 10 wt % dilution in water at room temperature). It was important during this stage to maintain the pH above 7 in order to avoid any acidic hydrolysis of ketal to ketone. Upon addition of H₃PO₄ (85%) aq. solution, there was formation of a white precipitate.

The obtained orange crude suspension (101.7 g) was then filtered on Celite in order to remove phosphate salts and the solid was washed several times with ethyl acetate. The solvent of the filtrate was then removed under vacuum (60° C., 30 mbar) giving 99.34 g of an orange oil. At this stage, the product still contained some inorganic salts.

The oil was then finally filtered on silica gel using 1 L of ethyl acetate as an eluent (containing 0.1 wt % of Et₃N) in order to remove traces of inorganic salts and for discoloration. The solvent was then evaporated under vacuum (80° C., 15 mbar) to afford the final product as a pale yellow oil (m=86.93 g) corresponding to an isolated yield of 79%.

¹H NMR (CDCl₃, 400 MHz) δ (ppm): 4.3-4.15 (m, 1H), 4.09-4.05 (m, 1H), 3.47-3.36 (m, 1H), 3.07 (s, 3H), 2.92 (s, 3H), 2.49-2.42 (m, 2H), 1.96-1.89 (m, 2H), 1.34 (s), 1.31 (s) (total of both diastereoisomers=3H), 1.23 (d), 1.22 (d) (sum of integrals=3H). ¹³C NMR (CDCl₃, 101 MHz) δ (ppm): 175.51, 175.43, 110.97, 110.84, 74.2, 73.33, 72.4, 72.1, 37.95, 36.28, 35.96, 35.73, 29.24, 28.95, 25.6, 24.64, 19.09, 18.59.

### Example 2

### Solubility of Different Mono-Fungicides

In Example 2, the solubility of the fungicides was measured in 3-(2,4-dimethyl-1,3-dioxolan-2-yl)-N,N-dimethylpropanamide (Example 1) as well as prior art solvents at 24° C. and at 0° C. after seeding. The concentration of the fungicides (w/v) was increased in 5% intervals and the highest still soluble concentration is stated in the table below.

TABLE 1
Maximum solubility (w/v) of different fungicides in the solvent of Example 1
and prior art solvents at 24° C. (and at 0° C. after seeding in parentheses).

| | | | Rhodiasolv ® | | Rhodiasolv ® | |
|---|---|---|---|---|---|---|
| Fungicide | Example 1 | Cyrene ™ | PolarClean | | ADMA10 | |
| Prothioconazole | 50% (50%) | 10% (10%) | 50% | (50%) | 40% | (40%) |
| Tebuconazole | 30% (30%) | 10% (10%) | 30% | (30%) | 25% | (20%) |
| Azoxystrobin | 10% (10%) | 20% (20%) | 15% | (5%) | 2% | (2%) |
| Trifloxystrobin | 25% (25%) | 30% (25%) | 25% | (25%) | 10% | (10%) |
| Prochloraz | 60% (50%) | 70% (55%) | 60% | (45%) | 45% | (40%) |
| Saflufenacil | 20% (20%) | 10% (10%) | 25% | (25%) | 10% | (10%) |
| Difenoconazole | 35% (35%) | 40% (40%) | 30% | (30%) | 15% | (15%) |
| Pyraclostrobin | 45% (45%) | 45% (45%) | 45% | (45%) | 15% | (10) |

### Example 3

### Solubility of Different Combi-Fungicides:

In Example 3, the solubility of the fungicide combinational formulations was measured in the ketal-amide derivative of Example 1 (3-(2,4-dimethyl-1,3-dioxolan-2-yl)-N,N-dimethylpropanamide) as well as prior art solvents at 24° C. It can be seen from Table 2 that the ketal-amide of Example 1 is an efficient solvent for combinational pesticide formulations. In addition, the solubility tests show that the ketal-amide of Example 1 is a better solvent than Rhodiasolv® PolarClean and Rhodiasolv® ADMA 10 for specific combinations, such as azoxystrobin/fluxapyroxad.

TABLE 2
Solubility of different combinational fungicide formulations (“Combi”) at 24° C.

| | Concentration | | | | |
|---|---|---|---|---|---|
| | (A/T, A/P or | | | Rhodiasolv ® | Rhodiasolv ® |
| Combi | A/F) in % w/v | Example 1 | Cyrene ™ | PolarClean | ADMA 10 |
| A/T | 7.5:20 | l | n | l | n |
| A/T | 15:20 | n | n | n | n |
| A/P | 10:15 | l | l | l | n |
| A/P | 15:20 | n | n | n | n |
| A/F | 9:6 | l | l | l | n |
| A/F | 12:8 | l | l | n | n |

“A” denotes azoxystrobin,
“T” denotes tebuconazole,
“P” denotes prothioconazole,
“F” denotes “fluxapyroxad”,
“l” denotes a limpid and clear solution,
“n” denotes non-solubility

### Example 4

### Solubility of Urease Inhibitor NBPT

The solubility of NBPT was measured in NBP, Cyrene™ and Rhodiasolv® PolarClean as well as the solvent of Example 1 at 24° C. and at −5° C. The results are depicted in Table 3 below. As can be seen, the solvent of Example 1 is an excellent solvent for dissolving NBPT. The use of Cyrene and NBP on the other hand led to a yellow discoloration even at low concentrations and the resulting solution was highly viscous even at 35% (w/v), thus making the resulting solutions difficult to use and further process. In addition, it can be seen that the ketal-amide solvent of Example 1 is significantly better for solubilizing NBPT than Rhodiasolv® PolarClean. Without being bound to theory, it is believed that the increased solubility in the ketal-amide solvent of Example 1 could be due a favourable enthalpy driven solubilization due to H-bond formation of the hydrogens groups of the amide functional groups of NBPT with the ketone functional group of the solvent.

TABLE 3
Maximum solubility (w/v) of NBPT in the solvent
of Example 1, NBP and Cyrene ™ at 24°
C. (and at −5° C. in parentheses).

| | | | | Rhodiasolv ® |
|---|---|---|---|---|
| | Example 1 | NBP | Cyrene ™ | Polarclean |
| Solubility | 40% (35%) | 50% (45%) | 55% (55%) | <30% |
| of NBPT | | | | |

### Example 5

### Solubility of Different Polymers

The ketal-amide of levulinic acid of Example 1 was tested as a NMP replacement as a solvent for dissolving polymers. The results are stated in Table 4 below. As can be seen, the ketal-amide of levulinic acid of Example 1 is a suitable replacement for the solvent NMP and can be used for the manufacturing of battery electrodes for solid-state battery or Li-ion battery or for coatings or to produce anti-fouling membranes for a wide range of filtration applications. PVDF and Technoflon® are polymer binders that are usually dissolved in NMP (or DMF and DMAc) and then used to dispense a slurry of a carbon material and the dissolved binder, also known as wet coating, onto a substrate material: the electrode. The use of the ketal-amide of levulinic acid instead of NMP during the manufacturing of electrodes, such as electrodes for car batteries, is environmentally friendly.

PVDF (Solef® 1015) is also used to make micro- and ultrafiltration membranes for a wide range of filtration applications by the NIPS process (Non-Solvent Induced Phase Separation). In NIPS, a polymer solution film is immersed in a non-solvent bath (water), inducing phase separation of the film into a polymer-rich phase that becomes the membrane matrix and a polymer-poor phase that becomes the membrane pores. N-methyl-2-pyrrolidone (NMP) is used as the solvents medium because it could successfully dissolve PVDF. The use of the ketal-amide of levulinic acid instead of NMP during the manufacturing of the membranes, is environmentally friendly.

Polyamide-imides PAI are used as high performance coating materials in both automotive industry and high-end household appliances. The main need here is to find good solvents to realize the coating formulation in liquid form. NMP, DMF and DMAC are known as good solvents of such polymers. The use of the ketal-amide of levulinic acid instead of NMP during the manufacturing of the membranes, is environmentally friendly.

TABLE 4
Solubility of different polymers (PVDF (Polyvinylidene
fluoride) Solef ® polymers, Tecnoflon ® fluoroelastomers
and Torlon ® PAI (Poly-Amide-Imide)) in
the ketal-amide of levulinic acid of Example 1.

| | Solubility at 5 | |
|---|---|---|
| Polymer | wt.-% at 70° C. | Relevant application |
| PVDF Solef ® 5130 | Yes | Electrode for battery |
| PVDF Solef ® 75040 | Yes | Electrode for battery |
| PVDF Solef ® 6030 | Yes | Electrode for battery |
| PVDF Solef ® 5140 | Yes | Electrode for battery |
| PVDF Solef ® 1015 | Yes | Anti-fouling membranes |
| | | for filtration applications |
| Tecnoflon ® NH100HS | Yes | Solid battery |
| Tecnoflon ® NH | Yes | Solid battery |
| Tecnoflon ® N90HS | Yes | Solid battery |
| Tecnoflon ® N935 | Yes | Solid battery |
| Torlon ® PAI | Yes | Coating |

### Example 6

### Synthesis of 3-(4-(hydroxymethyl)-2-methyl-1,3-dioxolan-2-yl)-N,N-dimethylpropanamide

The reaction is conducted in carefully dry vessels and under an inert nitrogen atmosphere.

In a carefully dried 1 L double-jacketed reactor equipped with a mechanical stirrer (propeller with 3 inclined plows), baffles, a temperature probe and a cooler (5° C.) are added at room temperature:

86.8 g of ethyl glycerol-ketal levulinate (0.40 mole, 1 eq.), obtainable following the protocol described in example 6 of WO 2012/018939, by using methanesulfonic acid as catalyst instead of 2-Naphtalenesulfonic acid,

397.7 mL of DMA/MeOH solution (308.2 g, 2M, 11 wt %, 0.795 moles, 2 eqs.).

The solution is then allowed to stir (650 rpm) at room temperature and 0.678 g of solid NaOMe (0.012 mole, 3 mol %) is added into the solution in one shot. The solution is then allowed to stir at 50° C. (600 rpm) during 3 h00. At this stage, 1H NMR analysis showed that the ester conversion level to the desired amide product reached 20 mol %. In order to speed up the reaction kinetic, an additional amount of solid sodium methylate is added into the solution (1.58 g, 0.028 mole, 7 mol %). The mixture is then stirred at 50° C. during 48 h00 allowing to reach 96 mol % of ester conversion to the amide.

The volatiles (DMA, MeOH and ethanol) are then removed under vacuum (60° C., 2 mbar, 307.2 g of collected distillate) and the catalyst in the residue is carefully neutralized thanks to the addition at room temperature of 1.834 g of H₃PO₄ (aqueous solution 85 wt %, 0.016 mole, 0.4 eq. vs. NaOMe) in order to decrease down the pH of the mixture between 7.5 and 8.5 (final pH=7.41, measured after 2 wt % dilution in water at room temperature). It is important during this stage to maintain the pH above 7 in order to avoid any acidic hydrolysis of the ketal to the ketone. Upon addition of H₃PO₄ (85%) aq. solution, no formation of a white precipitate is observed in that case.

The obtained residue is then heated at 70° C. under 13 mbar vacuum in order to remove all the volatiles, mainly methanol residual and ethanol and water that have been formed during the catalyst neutralization step. The crude is finally diluted with a minimum amount of ethyl acetate containing 0.1 wt % of triethylamine. Upon addition of ethyl acetate, phosphate salts precipitation is observed. The suspension is then filtered on silica gel and eluted using 1 L of ethyl acetate as an eluent (containing 0.1 wt % of Et₃N) in order to remove traces of inorganic salts and for discoloration. The solvent is then evaporated under vacuum (80° C., 10 mbar) to afford the final product as a viscous yellow oil (m=75 g) corresponding to an isolated yield of 85%.

1H NMR analysis shows that the desired ketal amide content is 98.5 wt % and the product additionally contains 1.5 wt % of N,N-dimethyl levulinamide as by-product (Acid Value=4.2 mg(KOH)/g, Karl-Fisher: 0.2 wt % H₂O).

1H NMR (CDCl₃, 400 MHz) δ (ppm): 4.24-4.11 (m, 1H), 4.08-4.04 (m, 1H), 3.77-3.7 (m, 1H), 3.63-3.54 (m, 2H), 3.07 (s, 3H), 2.92 (s, 3H), 2.52-2.42 (m, 2H), 1.99-1.91 (m, 2H), 1.36 (s), 1.32 (s) (total of both diastereoisomers=3H).

13C NMR (CDCl₃, 101 MHz) δ (ppm): 175.62, 175.37, 111.47, 111.25, 78.57, 77.75, 67.67, 67.57, 63.96, 63.67, 37.95, 35.96, 35.91, 35.31, 29.22, 28.83, 25.25, 24.23.

### Example 7

### Solubility of Different Mono-Fungicides

In Example 7, the solubility of the fungicides was measured in 3-(4-(hydroxymethyl)-2-methyl-1,3-dioxolan-2-yl)-N,N-dimethylpropanamide (Example 6) as well as prior art solvents at 24° C.

TABLE 5
Maximum solubility (w/v) of different fungicides in the
solvent of Example 6 and prior art solvents at 24° C.

| | | | Rhodiasolv ® |
|---|---|---|---|
| Fungicide | Example 6 | Cyrene ™ | ADMA10 |
| Prothioconazole | >30% | 10% | 40% |
| Tebuconazole | >20% | 10% | 25% |
| Azoxystrobin | 10% | 20% | 2% |
| Trifloxystrobin | >15% | 30% | 10% |
| Saflufenacil | >10% | 10% | 10% |
| Difenoconazole | >25% | 40% | 15% |
| Pyraclostrobin | >25% | 45% | 15% |

### Example 8

### Solubility of Urease Inhibitor NBPT

The solubility of NBPT was measured in the solvent of Example 6 at 24° C., in addition to the previous measures (Example 4) in NBP, Cyrene™ and Rhodiasolv® PolarClean. The results are depicted in Table 6 below. As can be seen, the solvent of Example 6 is very good solvent for dissolving NBPT. As mentioned in Example 4, the use of Cyrene and NBP on the other hand led to a yellow discoloration even at low concentrations and the resulting solution was highly viscous even at 35% (w/v), thus making the resulting solutions difficult to use and further process. In addition, it can be seen that the ketal-amide solvent of Example 6 is better for solubilizing NBPT than Rhodiasolv® PolarClean.

TABLE 6
Maximum solubility (w/v) of NBPT in the solvent of Example
6, NBP and Cyrene ™ at 24° C.

| | | | | Rhodiasolv ® |
|---|---|---|---|---|
| | Example 6 | NBP | Cyrene ™ | Polarclean |
| Solubility | >30% | 50% | 55% | <30% |
| of NBPT | | | | |

1. A compound of formula (I): 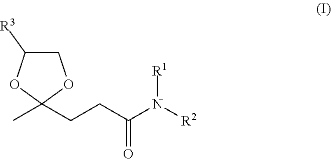
wherein
R¹ and R² are independently selected from —(C₁-C₆)alkyl, wherein —(C₁-C₆)alkyl comprises —(C₁)alkyl, —(C₂)alkyl, —(C₃)alkyl, —(C₄)alkyl, —(C₅)alkyl, and/or —(C₆)alkyl, optionally interrupted and/or substituted by a heteroatom or heteroatom-containing group, or R¹ and R² form with the nitrogen atom a heterocycle containing from 4 to 8 atoms; and
R³ is hydrogen, or —(C₁-C₄)alkyl, optionally interrupted and/or substituted by a heteroatom or heteroatom containing group;
with the proviso that when R¹ and R² are —CH₃, then R³ is not hydrogen.

2. The compound according to claim 1,
wherein R¹ and R² are independently selected from —(C₁-C₃)alkyl, wherein —(C₁-C₃)alkyl comprises —(C₁)alkyl, —(C₂)alkyl, and/or —(C₃)alkyl.

3. The compound according to claim 1,
wherein R³ is —(C₁-C₂)alkyl, wherein —(C₁-C₂)alkyl comprises —(C₁)alkyl and/or —(C₂)alkyl, optionally interrupted and/or substituted by a heteroatom.

4. The compound according to claim 1, wherein
R³ is —(C₁)alkyl, optionally substituted by a heteroatom.

5. The compound according to claim 1, wherein
R¹ and R² are independently selected from —(C₁-C₆)alkyl, and
R³ is —(C₁-C₄)alkyl, optionally interrupted and/or substituted by a heteroatom or heteroatom containing group.

6. The compound according to claim 1, wherein
R¹ and R² are independently selected from —(C₁)alkyl, —(C₂)alkyl, and —(C₃)alkyl, and
R³ is —(C₁-C₄)alkyl.

7. The compound according to claim 1, wherein
R¹ and R² are independently selected from —(C₁)alkyl, —(C₂)alkyl, and —(C₃)alkyl, and
R³ is —(C₁-C₂)alkyl.

8. The compound according to claim 7, wherein R¹, R² and R³ are —(C₁)alkyl.

9. The compound according to claim 1, wherein
R¹ and R² are independently selected from —(C₁)alkyl, —(C₂)alkyl, and —(C₃)alkyl, and
R³ is —CH₂—OH.

10. The compound according to claim 9, wherein R¹ and R² are —(C₁)alkyl

11. A method for producing the compound according to claim 1 from levulinic acid or an ester or salt thereof, comprising at least the following steps in any order:
(a) forming a ketal with a compound of formula R³—CH(OH)—CH₂OH
(b) forming an amide with an amine of formula HNR¹R²,
wherein R¹ to R³ are defined in claim 1.

12. The method according to claim 11, wherein step (a) is acid-catalyzed and step (b) is base-catalyzed.

13. The method according to claim 12, wherein step (b) further comprises neutralizing the base.

14. A composition comprising the compound according to claim 1.

15. The composition according to claim 14 that is at least part of a phytosanitary formulation, a cleaning formulation, a stripping formulation, a degreasing formulation, a lubricant or textile formulation, a coating formulation.

16. The composition according to claim 15 that is a phytosanitary formulation comprising:
a) a phytosanitary active compound;
b) the compound according to claim 1;
c) optionally at least one emulsifier;
d) optionally water.

17. The composition according to claim 15 that is a phytosanitary formulation comprising:
a) from 0.01 to 90% by weight of at least one agricultural active compound relative to the total weight of the phytosanitary formulation,
b) from 5 to 90% by weight of a compound according to claim 1, relative to the total weight of the phytosanitary formulation,
c) optionally, from 0.1 to 40% by weight of at least one said co-solvent, relative to the total weight of the phytosanitary formulation,
d) from 0.05 to 40% by weight of at least one surfactant, relative to the total weight of the phytosanitary formulation,
e) optionally, from 5 to 90% by weight of water, relative to the total weight of the phytosanitary formulation.

18. The composition according to claim 16, wherein the phytosanitary formulation comprises a fertilizer, a fertilizer stabilizer, a fungicide, an herbicide, an insecticide, an acaricide, an algicide, a molluscide, a miticide, a nematicide, a biocide or a rodenticide.

19. A method for providing a solvent, co-solvent and/or crystallization inhibitor or as a coalescing agent, the method comprising providing the compound according to claim 1 as the solvent, the co-solvent and/or the crystallization inhibitor or as the coalescing agent.

20. The method according to claim 19, wherein the providing comprises providing the compound in a phytosanitary formulation, as a solvent for at least one polymer, in coating applications, for the manufacture of membranes, for the manufacture of solid batteries, for the recycling of polymers, for the preparation, in solution, of polycondensates, for the cleaning of equipments in polymerization reactors, in household care formulations, for cleaning hard surfaces, or in the industrial sphere for degreasing manufactured products and/or for cleaning the manufactured products.

21.-23. (canceled)
